(19) United States
(12) Patent Application Publication
(10) Pub. No.: US 2009/0121159 A1
(43) Pub. Date: May 14, 2009

(54) CLUSTER E-BEAM LITHOGRAPHY SYSTEM

(75) Inventors: ARCHI HWANG, Hisnchu (TW); YI XIANG WANG, Fremont, CA (US); JACK JAU, Los Alto, CA (US); CHUNG-SHIH PAN, Palo Alto, CA (US); ZHONGWEI CHEN, San Jose, CA (US)

Correspondence Address: SAWYER LAW GROUP LLP, 2465 E. Bayshore Road, Suite No. 406, PALO ALTO, CA, 94303, US

(73) Assignee: HERMES-MICROVISION, INC., Hisnchu (TW)

(21) Appl. No.: 12/259,280

(22) Filed: Oct. 27, 2008

Related U.S. Application Data
(60) Provisional application No. 60/983,130, filed on Oct. 26, 2007.
(60) Provisional application No. 61/044,633, filed on Apr. 14, 2008.

(51) Int. Cl. A61N 5/00

(52) U.S. Cl. 250/492.3

(57) ABSTRACT

A hybrid lithography system is disclosed to achieve high throughput and high resolution of sub 32 nm lithography. The hybrid system contains an optical lithographer for expose pattern area where features above 32 nm, and a cluster e-beam lithography system for expose pattern area where features is sub 32 nm.

### CROSS-REFERENCES TO RELATED APPLICATIONS

Under 35 USC §120, this application is a Non-Provisional application and is related to co-pending U.S. Patent Application Ser. No. 60/983130, filed on Oct. 26, 2007, entitled “CLUSTER E-BEAM LITHOGRAPHY SYSTEM”; to U.S. Patent Application Ser. No. 61/044633, filed on Apr. 14, 2008, entitled “CLUSTER E-BEAM LITHOGRAPHY SYSTEM”, all of which are incorporated herein by reference.

### BACKGROUND OF THE INVENTION

This invention relates to an advanced lithography system to gain high throughput and high resolution in semiconductor lithography practice. More specifically, embodiments of the present invention provide a hybrid system that combines optical and e-beam lithography (EBL) system to achieve the object. Merely by way of example, the present invention has been used onto smallest feature lithograph such as gate, AA and contact, but it would be recognized that the invention has a much broader range of applicability.

Fabrication of semiconductor device such as logic and memory device may include processing wafer through various semiconductor processing tools. As feature size continuous shrink from 45 nm to 32 nm, conventional high throughput optical lithography system does not have high enough resolution for sub 32 nm nodes. The conventional e-beam lithography system has high resolution but has very low throughput during lithography practice.

Therefore, an improved system to achieve both high resolution and high throughput is desired.

### BRIEF SUMMARY OF THE INVENTION

This invention relates to an advanced lithography system to gain high throughput and high resolution in semiconductor lithography practice. More specifically, embodiments of the present invention provide a hybrid system that combines optical and e-beam lithography system to achieve the object. Merely by way of example, the present invention has been used onto smallest feature lithograph such as gate, AA and contact, but it would be recognized that the invention has a much broader range of applicability.

An object of the present invention is to provide a hybrid lithography system that combines one optical lithographer to expose larger pattern area and a cluster e-beam lithography system to expose pattern area where request higher resolution.

### BRIEF DESCRIPTION OF THE DRAWINGS

FIG. 1 is a simplified diagrammatic representation of a cluster e-beam lithography system according to an embodiment of the present invention.

FIG. 2 is a simplified diagrammatic representation of a functional flowchart of EBL system control center according to an embodiment of the present invention.

### DETAILED DESCRIPTION OF THE INVENTION

This invention relates to an advanced lithography system to gain high throughput and high resolution in semiconductor lithography practice. More specifically, embodiments of the present invention provide a hybrid system that combines optical and e-beam lithography system to achieve the object. Merely by way of example, the present invention has been used onto smallest feature lithograph such as gate, AA and contact, but it would be recognized that the invention has a much broader range of applicability.

As explained above, the e-beam lithographer is characterized with high resolution and low throughput; the optical lithographer has high throughput but not high enough resolution for sub 32 nm nodes in a semiconductor device. Moreover, a hybrid system with a cluster e-beam lithography system and an optical lithography system can take advantages of both. More specifically, the cluster e-beam lithography system exposes for small features and the optical lithography system exposes for larger patterns.

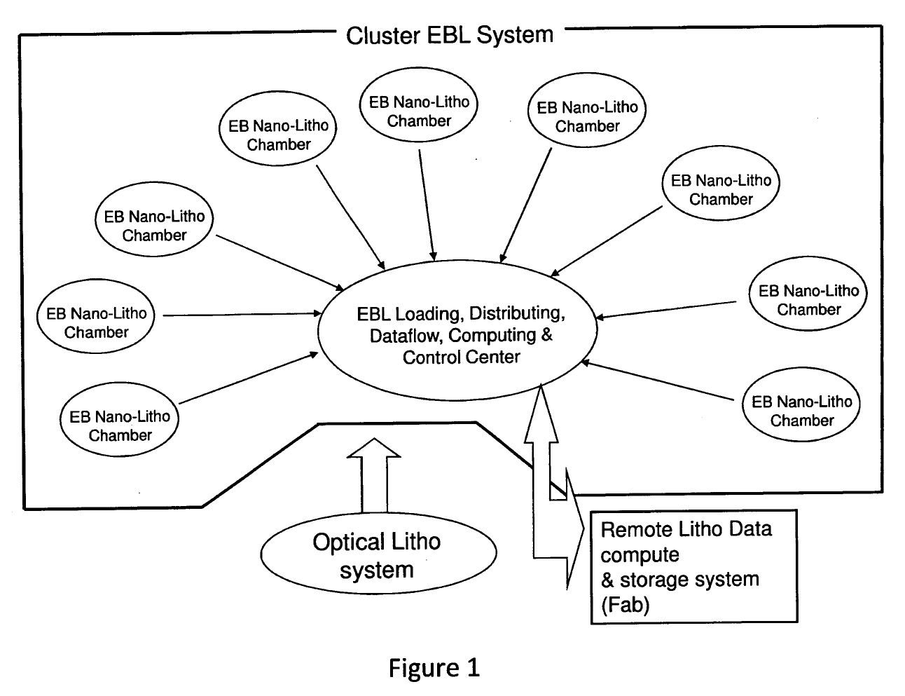

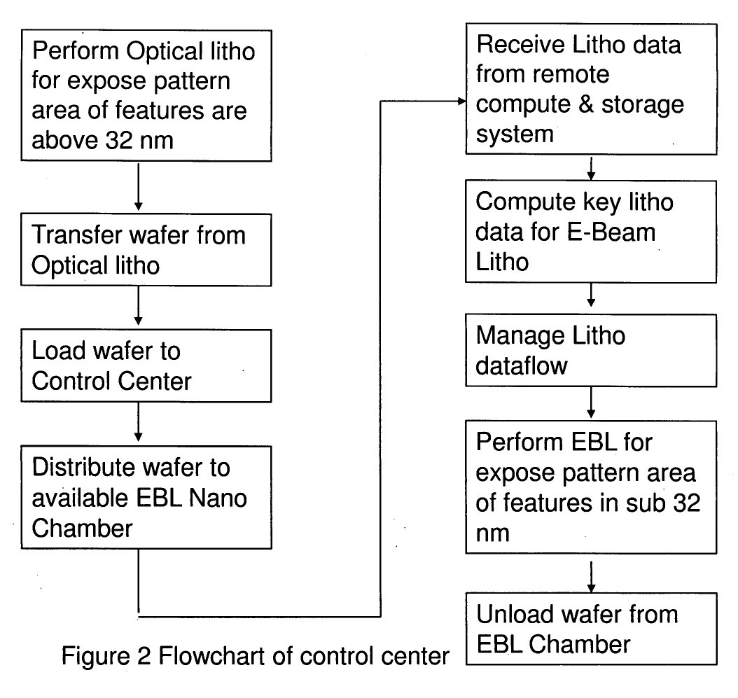

FIG. 1 is a simplified diagrammatic representation of a hybrid lithography concept to achieve high throughput and high resolution lithograph for sub 32 nm patterns. The optical lithography system exposes the larger feature of the pattern. Then pass the wafer to the cluster e-beam lithography system to expose the sub 32 nm features. A cluster e-beam system is configured more than one e-beam lithographer tool to mach the throughput debt; a control center that managing wafer loading/unloading, wafer distributing, dataflow, data processing and system control; a litho data compute and storage system that compute and storage key litho parameters. The litho data compute and storage system may locate at a remote site per customer request. The FIG. 2 illustrates the functional flowchart of a cluster EBL system.

What is claimed is:

1. A hybrid lithography system to achieve high throughput and high resolution of sub 32 nm lithography, the hybrid lithography system comprising:
an optical lithographer for expose pattern area where features are above 32 nm; and
a cluster e-beam lithography system for expose pattern area where features are sub 32 nm.

2. The cluster e-beam lithography system of claim 1, wherein at least one e-beam nano-litho chamber is configured to match the throughput of optical lithographer.

3. The cluster e-beam lithography system according to claim 1, further comprising an e-beam lithography control center to managing the wafer loading, unloading, wafer distributing, and dataflow computing.

4. The cluster e-beam lithography system according to claim 1, further comprising a litho data compute and storage system for computing and storing key litho parameters; wherein the data compute and storage system may be located at a remote site from the main cluster e-beam lithography system.
